(19) United States
(12) Patent Application Publication
(10) Pub. No.: US 2013/0057720 A1
(43) Pub. Date: Mar. 7, 2013

(54) ELECTRONIC DEVICE

(71) Applicants: Kohei KAWAJI, Tokyo (JP); Masakazu SEKIGUCHI, Kawasaki-shi (JP)

(72) Inventors: Kohei KAWAJI, Tokyo (JP); Masakazu SEKIGUCHI, Kawasaki-shi (JP)

(73) Assignee: NIKON CORPORATION, Tokyo (JP)

(21) Appl. No.: 13/610,364

(22) Filed: Sep. 11, 2012

Related U.S. Application Data
(63) Continuation of application No. PCT/JP2010/006823, filed on Nov. 22, 2010.

(30) Foreign Application Priority Data
Mar. 15, 2010 (JP) 2010-058268

(51) Int. Cl. H04N 5/228

(52) U.S. Cl. 348/222.1; 348/E05.031

(57) ABSTRACT

The number of control parameters to be set has increased in electronic devices, particularly those with complex systems, and with automatic control parameter setting, even a small change in conditions can result in control result that differs greatly from what the user desires, which results in poor operability and worse output. Therefore, provided is an electronic device comprising a processing section that performs processing and a control section that changes the processing by the processing section upon receiving a change in biometric information of a user caused by the processing of the processing section.

The Contents of the following Japanese patent applications are incorporated herein by reference:

NO.2010-058268 filed on Mar. 15, 2010, and

PCT/JP2010/006823 filed on Nov. 22, 2010.

### TECHNICAL FIELD

The present invention relates to an electronic device.

### BACKGROUND ART

An image capturing device is known that estimates the emotional state of a photographer by detecting biometric information of the photographer, and assists with the image capturing operation based on the estimated emotional state. For example, in response to a high emotional level, the camera shake correction gain is adjusted to improve the tracking characteristics of the correction lens.

### Patent Document 1: Japanese Patent Application Publication No. 2009-210992

### DISCLOSURE OF THE INVENTION

### Problems to be Solved by the Invention

The environment in which a mobile hand-held electronic device is used can usually be expected to change with time. Conventionally, it is preferable that control parameters be automatically set according to the environment in which the electronic device is used, in order to provide the user with suitable operability and output. However, the automatic setting of the control parameters is often not to the liking of the user. The number of control parameters to be set has increased in electronic devices, particularly those with complex systems, and with automatic control parameter setting, even a small change in conditions can result in control result that differs greatly from what the user desires, which results in poor operability and worse output.

### Means for Solving the Problems

The electronic device according to an aspect of the present invention comprises a processing section that performs processing and a control section that changes the processing by the processing section upon receiving a change in biometric information of a user caused by the processing of the processing section.

The summary clause does not necessarily describe all necessary features of the embodiments of the present invention. The present invention may also be a sub-combination of the features described above.

### BRIEF DESCRIPTION OF THE DRAWINGS

FIG. 1 is a cross-sectional view of components in a camera system.

FIG. 2 is an overhead perspective view of the camera system.

FIG. 3 shows a first state in which the photography lens is held with the left hand.

FIG. 4 shows a second state in which the photography lens is held with the left hand.

FIG. 5 shows a camera-side biosensor section provided on the camera body.

FIG. 6 shows configurations of a heart rate detection apparatus and a pulse detection apparatus.

FIG. 7 is a block diagram of the camera system.

FIG. 8 is a flow chart of the autofocus control.

FIG. 9 is a flow chart of the image capturing control.

### BEST MODE FOR CARRYING OUT THE INVENTION

Hereinafter, some embodiments of the present invention will be described. The embodiments do not limit the invention according to the claims, and all the combinations of the features described in the embodiments are not necessarily essential to means provided by aspects of the invention.

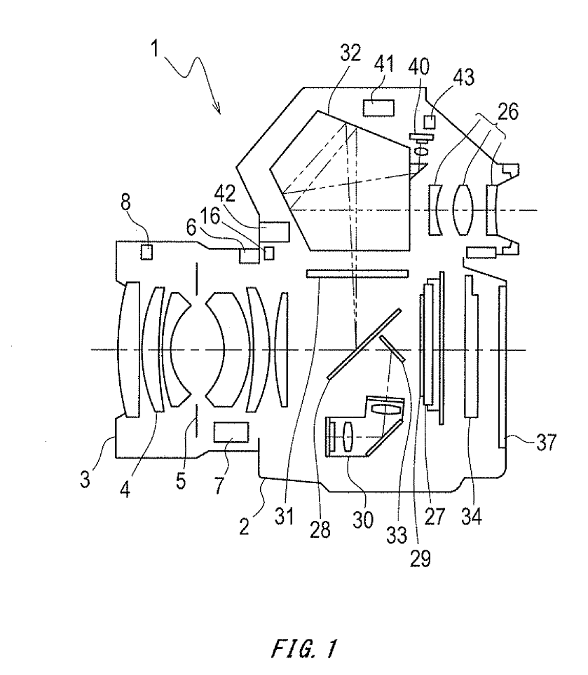

FIG. 1 is a cross-sectional view of a camera system 1 according to an embodiment of the present invention. In the present embodiment, the camera system 1 is described as an example of a mobile device. The camera system 1 is a single-lens reflex camera with an exchangeable lens, and functions as an image capturing apparatus resulting from the combination of a camera body 2 and an exchangeable photography lens 3.

The photography lens 3 includes a lens group 4 that has a focus lens, a zoom lens, and an image-stabilizing lens, a diaphragm 5, an angular velocity sensor 6 for detecting camera shake of the camera system 1, and a drive apparatus, not shown, that chives the lens group 4. The angular velocity sensor 6 detects vibration on at least two axes orthogonal to the optical axis. The drive apparatus may include a plurality of motors, such as oscillating wave motors and VCMs, drives the focus lens in a direction of the optical axis, and drives the image-stabilizing lens in a different direction than the optical axis direction.

The photography lens 3 includes a lens CPU 7 that operates together with the camera body 2 to control the overall photography lens 3. The photography lens 3 includes a lens-side biosensor section 8 that detects the pressure with which the photography lens 3 is held, body temperature, amount of sweat, blood pressure, blood flow, and heart rate of the photographer, for example.

The camera body 2 includes a main mirror 28 that pivots between a reflecting position, which is a position for reflecting light from the photography lens 3 to a finder optical system 26, and a withdrawn position in which the main mirror 28 is withdrawn such that the light from the photography lens 3 is incident to an image capturing element 27, which is formed by CCD or CMOS elements. A portion of the main mirror 28 is a semi-transparent region, and the camera body 2 includes a sub-mirror 30 that reflects the light passed through this semi-transparent region to a focal point detection sensor 29. The sub-mirror 30 pivots together with the main mirror 28, and the sub-mirror 30 is also withdrawn from the path of the light when the main mirror 28 is at the withdrawn position. The focal point detection sensor 29 detects the focal point state of the incident light based on the phase difference.

The light reflected by the main mirror 28 at the reflecting position is guided to the finder optical system 26 through a focusing screen 31 and a pentaprism 32. The finder optical system 26 is formed by a plurality of lenses, and the photographer can use the finder optical system 26 to check the field being captured.

A portion of the light passed by the pentaprism 32 is guided to a photometric sensor 40. The photometric sensor 40 measures light incident to each of a plurality of regions of the photography lens 3 to generate a brightness distribution of the field being captured. A GPS (Global Positioning System) module 41 is disposed above the pentaprism 32, and the camera system 1 receives a signal from a GPS satellite to acquire position information. The camera body 2 includes a microphone 42 that acquires sound in the field being captured and is positioned in a manner to not interfere with the photography lens 3 when the photography lens 3 is mounted on a mount section, and also includes a speaker 43 near the finder optical system 26.

When the main minor 28 is at the withdrawn position, the light from the photography lens 3 is incident to the image capturing element 27 through the low-pass filter 33. An image capturing substrate 34 is disposed near the image capturing element 27, and a rear surface monitor 37 is provided behind the image capturing substrate 34 to face outward.

The camera body 2 includes a camera-side biosensor section 16 that detects the pressure with which the camera body 2 is held, body temperature, amount of sweat, blood pressure, blood flow, or heart rate of the photographer, for example, at a position where a finger of the right hand of the photographer touches the camera-side biosensor section 16. The specific configuration and arrangement of the camera-side biosensor section 16 is described further below.

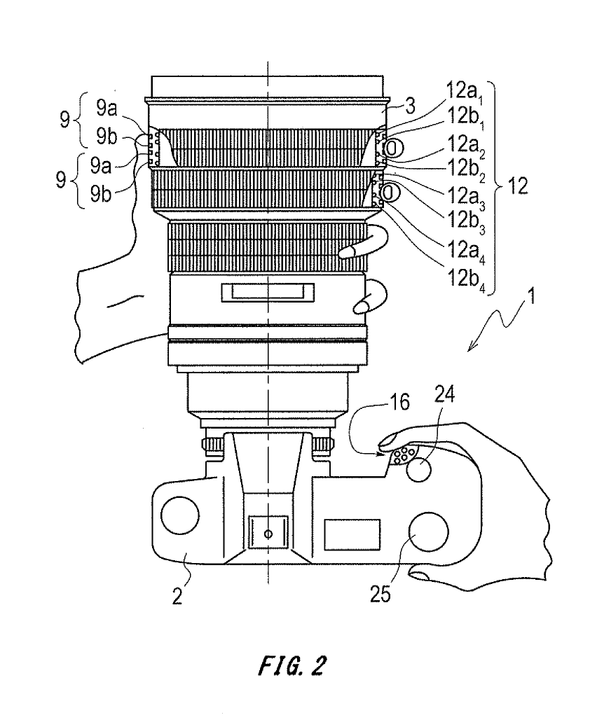

FIG. 2 is a perspective view of the top of the camera system 1 according to the present embodiment. Specifically, FIG. 2 shows a state in which the operator holds the photography lens 3 with the left hand while holding the pulse camera body 2 with the right hand. As described above, the photography lens 3 includes lens-side biosensor sections 8 that detect the pressure with which the photography lens 3 is held, body temperature, amount of sweat, blood pressure, blood flow, or heart rate of the photographer, for example. The lens-side biosensor sections 8 are positioned to be touched by the fingers or the palm of the left hand of the photographer.

In FIG. 2, a heart rate detection apparatus 9 and a pulse detection apparatus 12 are shown as a portion of the lens-side biosensor sections 8. The heart rate detection apparatus 9 includes a plurality of electrode sections each formed by a reference electrode 9a and a detection electrode 9b provided at a distance from each other, and the heart rate detection apparatus 9 detects the heart rate of the photographer. The pulse detection apparatus 12 is formed by a plurality of light emitting sections 12a (12a₁ to 12a₄) and corresponding light receiving sections 12b (12b₁ to 12b₄) arranged in an alternating manner, and the pulse detection apparatus 12 detects the pulse of the photographer. As described further below, the pulse detection apparatus 12 is used to measure the blood flow and blood pressure of the photographer.

As described above, the camera body 2 includes the camera-side biosensor section 16 at a location to be touched by a finger on the right hand of the photographer. When the photographer holds the camera body 2, the thumb on the right hand is positioned on the rear surface of the camera body 2 and the pointer finger is positioned near a release SW 24, and therefore these fingers are distanced from the other three fingers positioned on the grip section. As a result, the camera-side biosensor sections 16 are distanced from each other and provided at a rear camera position corresponding to the thumb of the right band, a release SW 24 proximate position corresponding to the pointer finger, and a front camera position near the grip portion that corresponds to the other three fingers. The camera-side biosensor section 16 corresponding to the pointer finger may be provided on the surface of the release SW 24.

In the camera body 2, at least one of the front camera position where the camera body 2 is held by the thumb and three fingers excluding the pointer finger on the right hand and the rear camera position where corresponding to the thumb of the right hand serves as a holding portion for holding the camera body 2. Furthermore, a plurality of operating SWs are provided on the rear surface of the camera body 2, and these operating SWs are operated by the right thumb. An image capturing mode SW 25 for setting the image capturing mode is provided on the top surface of the camera body 2.

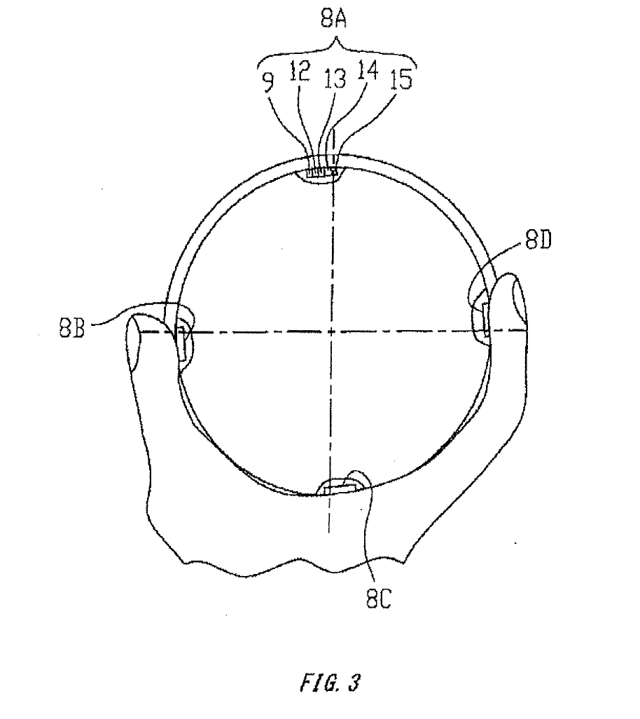

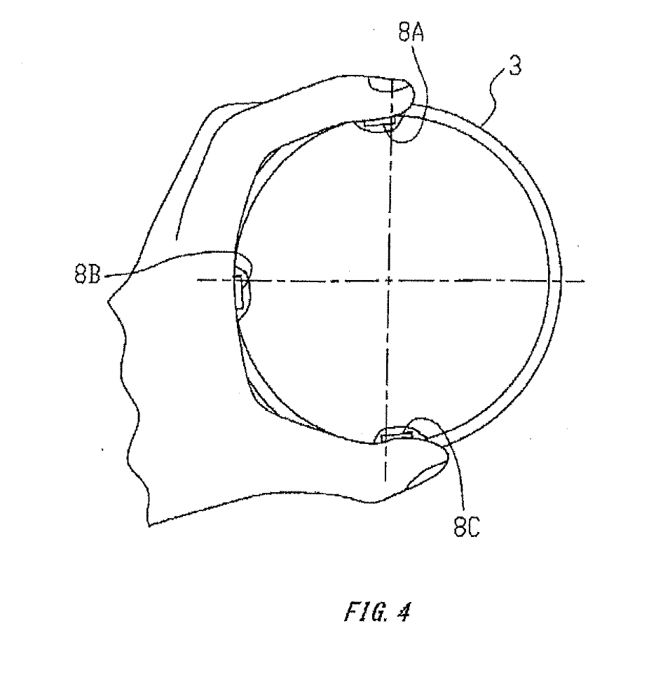

FIG. 3 shows a first state in which the photography lens 3 is held by the left hand. In the first state, the back of the left hand is positioned at the bottom when holding the photography lens 3. FIG. 4 shows a second state in which the photography lens 3 is held by the left hand. In the second state, the back of the hand is positioned on the left side when holding the photography lens 3.

When the photographer holds the photography lens 3 and performs a zoom operation or manual focus operation, the thumb of the left hand is distanced from the other norm. Furthermore, the method for holding the photography lens changes depending on the photographer or the photography conditions, such as horizontally oriented photography or vertically oriented photography. Therefore, the plurality of lens-side biosensor sections 8 (8A to 8D) are provided in the circular periphery of the photography lens 3.

Specifically, the lens-side biosensor sections 8 are disposed at least at one of a zoom operation position and a manual focus operation position, and are disposed apart from each other at a position corresponding to the thumb of the left hand and a position corresponding to a finger other than the thumb. More specifically, the lens-side biosensor sections 8 are disposed at positions where zoom operation rubber or focus operation rubber is disposed, and are disposed in a manner to contact the left hand or face the left hand.

The lens-side biosensor section 8A further includes a sweat sensor 13 that detects the amount of sweat of the photographer, a temperature sensor 14 that detects the body temperature of the photographer, and a pressure sensor 15 that detects the pressure with which the photographer holds the photography lens 3, in addition to the heart rate detection apparatus 9 and the pulse detection apparatus 12 described above.

The lens-side biosensor sections 8B to 8D each include a heart rate detection apparatus 9, a pulse detection apparatus 12, a sweat sensor 13, a temperature sensor 14, and a pressure sensor 15, in the same manner as the lens-side biosensor section 8A. In this way, biometric information can be detected from the palm of the left hand by providing the lens-side biosensor sections 8A to 8D on the circular periphery of the photography lens 3.

In the present embodiment, the plurality of lens-side biosensor sections 8A to 8D are provided according to the zoom operation position and the manual forces operation position, for example, but other lens-side biosensor sections 8 may be provided at positions other than those described above, as long as the positions allow for detection of the biometric information when the method of holding the photography lens 3 changes due to a different photographer or different photography state, for example. Furthermore, since the thumb of the left hand does not exert a large force for holding the photography lens 3, the lens-side biosensor sections 8B and 8C may have the pressure sensor 15 corresponding to the thumb of the left hand omitted therefrom. Similarly, if a high degree of detection precision is not needed for the lens-side biosensor sections 8, the number of components in the photography lens 3 can be reduced by omitting sensors from the position corresponding to the left thumb. Furthermore, the lens CPU 7 may control the light to be emitted from the light emitting section 12a of the pulse detection apparatus 12 only when a finger is in contact with the pulse detection apparatus 12.

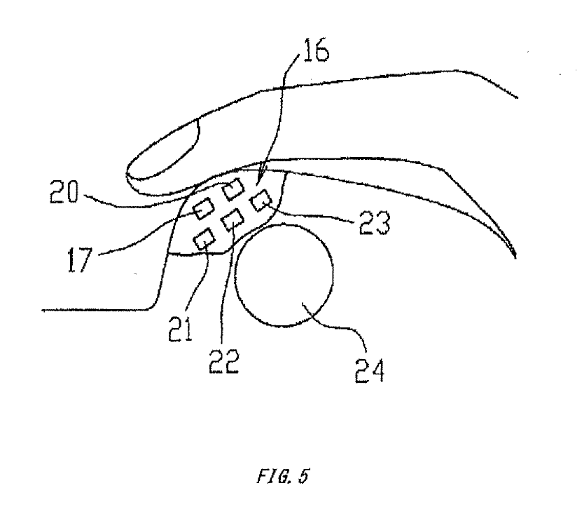

FIG. 5 shows the camera-side biosensor section 16 provided near the release SW 24 of the camera body 2. As shown in FIG. 5, the camera-side biosensor section 16 includes a heart rate detection apparatus 17, which has the same configuration as the heart rate detection apparatus 9, and a pulse detection apparatus 20, which has the same configuration as the pulse detection apparatus 12. The camera-side biosensor section 16 also includes a sweat sensor 21 that detects the amount of sweat of the photographer, a temperature sensor 22 that detects the body temperature of the photographer, and a pressure sensor 23 that detects the pressure with which the photographer holds the camera body 2. As described above, in addition to the position corresponding to the pointer finger of the right hand shown in FIG. 5, camera-side biosensor sections 16 are also provided at the rear camera position corresponding to the thumb and front camera position corresponding to the other three fingers, and each camera-side biosensor section 16 has the same configuration.

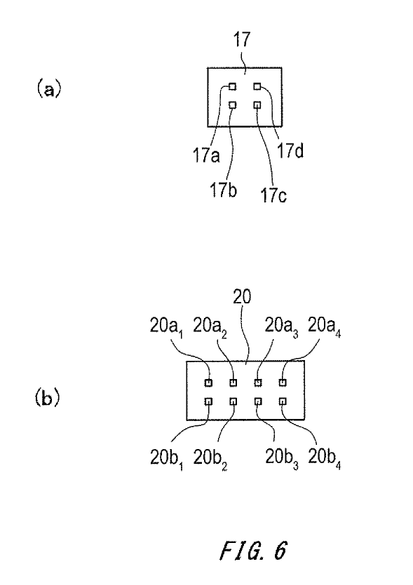

FIG. 6 shows the configurations of the heart rate detection apparatus 17 and the pulse detection apparatus 20 of the camera-side biosensor section 16. As shown in FIG. 6A, the heart rate detection apparatus 17 includes a plurality of electrode sections that each include a reference electrode 17a and a detection electrode 17b distanced from each other, and the heart rate detection apparatus 17 detects the heart rate of the photographer. As shown in FIG. 6B, the pulse detection apparatus 20 is formed by a plurality of light emitting sections 20a₁ to 20a₄ and corresponding light receiving sections 20b₁ to 20b₄ arranged in an alternating manner, and the pulse detection apparatus 20 detects the pulse of the photographer.

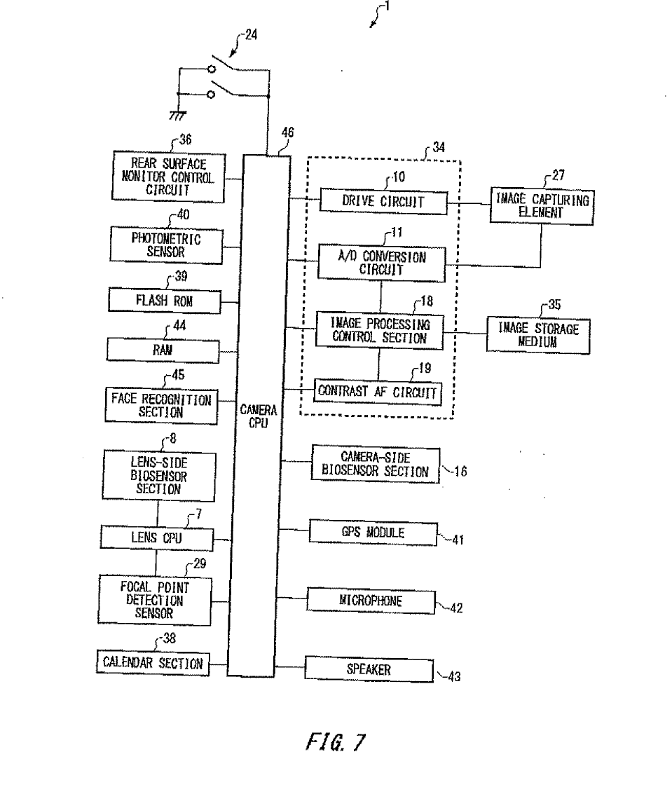

FIG. 7 is a block diagram of the camera system 1 according to the present embodiment. The image capturing substrate 34 includes a drive circuit 10 that drives the image capturing element 27, an A/D conversion circuit 11 that converts the output of the image capturing element 27 into a digital signal, an image processing control circuit 18 formed by ASIC, and a contrast AF circuit 19 that extracts a high frequency component of the signal from the image capturing element 27.

The image processing control circuit 18 applies image processing such as white balance adjustment, sharpness adjustment, gamma correction, and grayscale adjustment to the image signal that has been converted into a digital signal, and performs image compression such as JPEG on the image signal to generate an image file. The generated image file is stored in the image recording medium 35. The image recording medium 35 may be a storage medium such as a flash memory that can be attached to the camera body 2, or may be a storage medium such as an SSD (solid state drive) that is housed in the camera body 2.

The image signal that has undergone this image processing is displayed in the rear surface monitor 37 under the control of the rear surface monitor control section 36. If the image signal resulting from the image capturing is displayed for a prescribed time after the image capturing, a record-review display can be realized in which the photographer can view an image corresponding to the image file stored in the image recording medium 35. Furthermore, a live view display can be realized if a target image that is continuously photoelectrically converted by the image capturing element 27 is continuously displayed in the rear surface monitor 37 without being stored in the image recording medium 35. A moving image can be realized if the image processing control circuit 18 performs a moving image compression process such as MPEG or H.264 on the target image that is continuously photoelectrically converted by the image capturing element 27, and the resulting moving image is stored in the image recording medium 35. At this time, the sound in the field being captured is gathered by the microphone 42 and stored in synchronization with the moving image data. The frame rate of the generated moving image is set by selecting from among a plurality of frame rates, and may be 30 fps, for example.

The contrast AF circuit 19 extracts a high frequency component of the image capture signal from the image capturing element 27 to generate an AF evaluation image, and detects the focus lens position that maximizes the high frequency component. Specifically, a band-pass filter is used to extract a prescribed high frequency from the image signal received from the image processing control circuit 18, and a wave detection process such as peak-hold or integration is applied to generate the AF evaluation value signal. The generated AF evaluation value signal is output to the camera CPU 46.

The lens CPU 7 realizes the optical camera-shake correction by driving the image-stabilizing lens in the photography lens 3 in a direction differing from the optical axis direction, such that the camera-shake detected by the angular velocity sensor 6 is cancelled out. The camera-shake correction is not limited to this type of optical camera-shake correction, and the image capturing element 27 can be provided with a drive mechanism to perform an image capturing element drive camera-shake correction that cancels out the camera-shake by driving the image capturing element 27 in a direction differing from the optical axis direction. Furthermore, an electronic camera-shake correction can be used, whereby motion vectors between a plurality of images output by the image processing control circuit 18 are calculated and the camera shake is cancelled out by reading the images and controlling the position in a manner to cancel out the calculated motion vectors between the images. The optical camera-shake correction and the image capturing element drive camera-shake correction are particularly preferable when capturing still images, and can also be applied when capturing moving images. The electronic camera-shake correction is preferable when capturing moving images. These methods may be selected as needed or combined.

As described above, the photometric sensor 40 measures the brightness distribution of the capture field by measuring the light incident to each of a plurality of regions of the photography lens 3, and the measurement results are output to the camera CPU 46. In the camera CPU 46, an exposure value is calculated according to the selected photometric mode. The photometric mode can be selected from among a divisional photometric mode for obtaining a balance between light portions and dark portions, a center point photometric mode for exposing the center of the screen by an appropriate amount, and a spot photometric mode for exposing a selected focal point in a narrow range by an appropriate amount, for example.

The calendar section 38 includes a liquid crystal oscillator and an integrated circuit for keeping time, for example, and holds calendar information indicating year, month, day, and time. The camera CPU 46 can suitably detect information relating to the time from the calendar section 38. The GPS module 41 receives a signal from a GPS satellite and acquires information indicating the latitude, longitude, and altitude of the camera body 2. The camera CPU 46 can suitably detect information relating to the present position of the camera body 2 from the GPS module 41.

The flash ROM 39 is an EEPROM (Registered Trademark), and is a storage medium that stores programs causing the camera system 1 to operate, as well as various setting values and adjustment values. Specifically, the focal point detection sensor 29 stores AF adjustment data, AE adjustment data, data concerning the date and time of manufacture, setting history for the setting SW, and the like. Furthermore, the flash ROM 39 stores normal biometric information of the photographer. In the present embodiment, the flash ROM 39 stores pressure with which the photography lens 3 is held, pressure with which the camera body 2 is held, body temperature, amount of sweat, blood pressure, blood flow, and heart rate of the photographer as the biometric information.

The RAM 44 is a high-speed RAM such as a DRAM, that can expand the program stored in the flash ROM 39 to access the camera CPU 46 at high speed. The various setting values and adjustment values that are frequently referenced are copied from the flash ROM 39 to facilitate access from the camera CPU 46.

The face recognition section 45 determines whether a person's face is included as a subject in the captured image processed by the image processing control circuit 18. If a face is included, the face recognition section 45 detects the position and size of the face and outputs this information to the camera CPU 46. If there are a plurality of faces in the captured image, the face recognition section 45 can recognize a prescribed number of these faces. For example, when the release SW 24 is half-pressed during a record-review display, the face recognition section 45 performs facial recognition for the live view image being captured at that time. The camera CPU 46 displays a frame in the rear surface monitor control section 36 superimposed on the live view image to surround the recognized face, based on the position and size of the detected face.

The release SW 24 is a two-stage switch. When the photographer presses the release SW 24 half way, the camera CPU 46 uses the lens-side biosensor sections 8 and the camera-side biosensor sections 16 to begin detection of the biometric information of the photographer and to perform image capture preparation operations such as autofocus and light measurement. When the photographer fully presses the release SW 24, the camera CPU 46 starts the operation to capture a still image or a moving image.

The camera CPU 46 works together with the lens CPU 7 to control the overall camera system 1. In the present embodiment, the biometric information of the photographer is acquired based on the output of the lens-side biosensor sections 8 and the camera-side biosensor sections 16, and operations for assisting the camera system 1 are controlled. The following describes the acquisition of the biometric information of the photographer by the lens-side biosensor sections 8 and the camera-side biosensor sections 16.

First, the heart rate measurement will be described. As described above, the reference electrodes 9a and detection electrodes 9b of the electrode sections 9 are disposed at positions where the photography lens 3 is held by the left hand of the photographer, and the reference electrodes 17a and detection electrodes 17b of the heart rate detection apparatus 17 are disposed at positions where the camera body 2 is held by the right hand of the photographer. The difference between the potentials detected by the detection electrodes 9b and 16b is amplified by a differential amplifier, not shown, and output to the camera CPU 46. The body camera CPU 46 calculates the heart rate of the photographer based on the potential difference between the detection electrodes 9b and 16b.

If the photographer is not holding the photography lens 3, the left hand of the photographer is not in contact with the reference electrode 9a or the detection electrode 9b, and therefore the connection between the reference electrode 9a and the detection electrode 9b is open. When the connection between the reference electrode 9a and the detection electrode 9b is open, the lens CPU 7 determines that the photographer is not holding the photography lens 3. Similarly, when the connection between the reference electrode 17a and the detection electrode 17b of the heart rate detection apparatus is open, the camera CPU 46 determines that the photographer is not holding the camera body 2.

The following describes the blood pressure measurement. The pulse detection apparatuses 12 and 20 measure the blood pressure of the photographer. The pulse detection apparatus 12 and the pulse detection apparatus 20 have the same configuration, and therefore the following detailed description of the pulse measurement includes only the pulse detection apparatus 12. The pulse detection apparatus 12 emits infrared rays, for example, from the light emitting section 12a the infrared rats are reflected by the arteries in the fingers, and the reflected infrared rays are received by the light receiving section 12bm which is an infrared sensor, thereby detecting the pulse in the fingers. In other words, the pulse detection apparatus 12 detects the blood flow in a peripheral blood vessel. The camera CPU 46 calculates the blood pressure of the photographer based on the pulse received from the pulse detection apparatus 12. When it is determined that a finger of the photographer, e.g. the pinky, is not touching the photography lens 3 based on the outputs of the reference electrodes 9a and the detection electrodes 9b of the heart rate detection apparatuses 9, the lens CPU 7 prevents meaningless light output and the emission of stray light into the capture field by prohibiting the emission of light from the light emitting section 12a arranged to correspond to the pinky finger. Similarly, when it is determined that the thumb of the photographer is not touching the camera body 2 based on the outputs of the reference electrodes 17a and the detection electrodes 17b of the heart rate detection apparatus 17, the camera CPU 46 may prohibit light emission from the light emitting section 20a of the pulse detection apparatus 20.

The following describes the sweat measurement. Sweat can be detected by measuring the impedance of the hand. The sweat sensors 13 and 21 have a plurality of electrodes and detect sweat. A portion of these electrodes may also be used as the reference electrodes 9a and the reference electrodes 17a. A sweat sensor 13 is disposed in each of the lens-side biosensor sections 8A to 8D, but since sweat caused by emotional states such as happiness, excitement, or nervousness occurs in small amounts and in a short time, the lens-side biosensor sections 8B and 8C may be disposed at positions corresponding to the center of the palm, which creates more sweat than the fingers.

The following describes the temperature measurement. The temperature sensors 14 and 22 use thermistors with resistance values that change due to heat. There are different types of sweat including emotional sweat described above and thermal sweat for regulating body temperature, and these types of sweat can interfere with each other. Therefore, the camera CPU 46 can determine whether the sweat of the photographer is emotional sweat or thermal sweat based on the outputs of the sweat sensors 13 and 21 and the outputs of the temperature sensors 14 and 22. For example, the camera CPU 46 can determine the thermal sweat to be the sweat occurring when the temperature detected by the temperature sensor 22 is high and the sweat signal from the sweat sensor 21 is detected normally. Furthermore, the camera CPU 46 can determine the emotional sweat to be sweat occurring when the sweat signal from the sweat sensor 21 is irregular, and can therefore detect that the photographer is happy, excited, or nervous. If the temperature sensors 14 and 22 are not included, the body CPU 44 may judge whether the sweat signals from the sweat sensors 13 and 21 indicate emotional sweat or thermal sweat based on position information from the GPS module 41 or date and time information from the calendar section 38, for example. Furthermore, the lens CPU 7 may determine the sweat of the left hand to be emotional sweat or thermal sweat based on the output of the sweat sensor 13 and the output of the temperature sensor 14.

The following describes pressure measurement. The pressure sensor 15 is an electrostatic capacitance sensor, and measures a deformation amount caused by a pressing force when the photographer holds the photography lens 3. In the present embodiment, the pressure sensor 15 is disposed below operating rubber. The pressure sensor 23 is a similar electrostatic capacitance sensor, and measures the deformation amount caused by a pressing force when the photographer holds the camera body 2. The pressure sensors 15 and 23 may use distortion gauges or electrostriction elements, for example.

As described above, the camera CPU 46 works together with the lens CPU 7 to acquire biometric information of the photographer based on the output of the lens-side biosensor sections 8 and the camera-side biosensor sections 16 and to control assistance operations of the camera system 1. The following describes a specific example of control performed using the biometric information of the photographer.

### First Embodiment Example

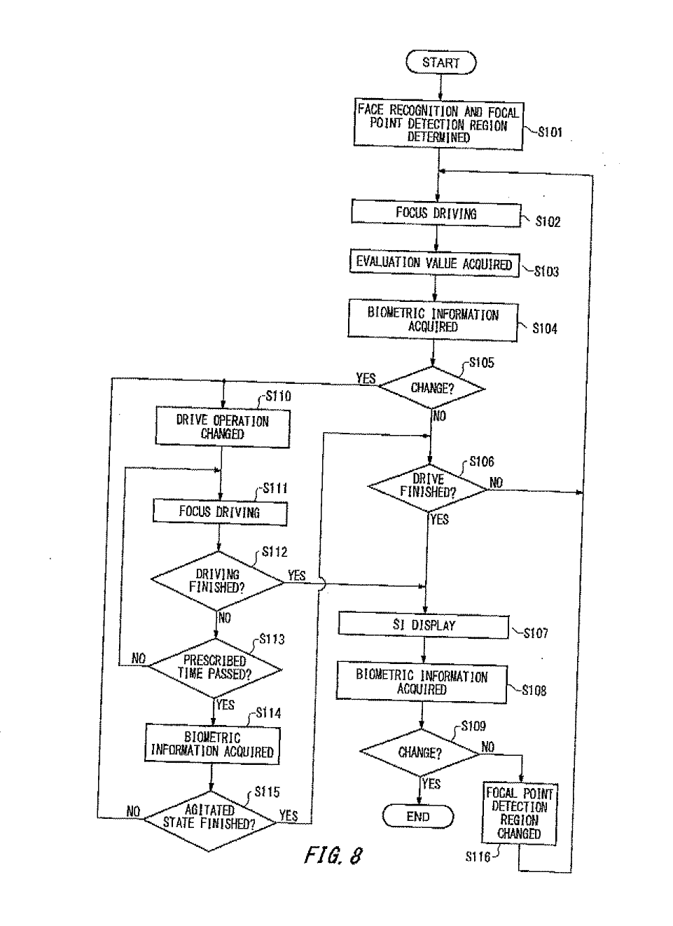

FIG. 8 is a flow chart of autofocus control as a first embodiment example of the present embodiment. In the present embodiment example, still image capturing is performed. The photographer turns ON the power supply of the camera system 1 and instructs record-review display to be performed by the rear surface monitor 37, for example, to begin the image capturing operation flow. The camera CPU 46 displays a live view image with adjusted exposure in the rear surface monitor 37, using the rear surface monitor control section 36. The exposure adjustment includes using a plurality of image signals from the image capturing element 27 and causing the average brightness value of one entire image to be within a prescribed range. Prior to when the live view display begins, the main mirror 28 is provided at the reflection position, the output from the photometric sensor 40 is obtained, and the camera CPU 46 may calculate the appropriate exposure corresponding to the photometric mode. The camera CPU 46 stands by until the photographer provides instructions for image capturing preparation by pressing the release SW 24 half way.

When the release SW 24 is pressed half way to designate the image capturing preparation, the process moves to step S101 and the face recognition section 45 uses the continuously input live view images to recognize whether a person's face is included as a subject. When the face of a subject is recognized, the face recognition section 45 detects the position and size of the recognized face and outputs this information to the camera CPU 46. The camera CPU 46 determines the focal point detection region to be a region determined by the position and size of the recognized face. If there are a plurality of faces recognized by the face recognition section 45, the camera CPU 46 determines the focal point region by selecting one of the faces according to conditions such as the face positioned nearest the center in the angle of field, the face positioned closest to the camera, or a face registered in advance. At this time, the camera CPU 46 may superimpose a yellow rectangular frame, for example, on the live view image to surround the region of the detected face. In particular, the camera CPU 46 preferably displays the region in a manner to be recognized by the photographer, such as by surrounding the region selected as the focal point detection region with a double line.

When the focal point detection region is determined, the process moves to step S102 and the camera CPU 46 begins driving the focus lens in a manner to focus in the determined focal point detection region. In the present embodiment example, the camera CPU 46 performs the contrast AF described above. The following describes the control performed to drive the contrast AF.

In the contrast AF, first, the contract AF circuit 19 cuts out the focal point detection region determined in step S101 from the received image signal, and extracts the high frequency component in this region to generate the AF evaluation value signal. The camera CPU 46 receives the AF evaluation value signal from the contrast AF circuit 19. The camera CPU 46 determines the drive direction of the focus lens predicted when the AF evaluation value signal is large by comparing the received AF evaluation signal with a previously acquired AF evaluation signal, and transmits a control signal to the lens CPU 7 to drive the focus lens in this direction. The lens CPU 7 receives the control signal from the camera CPU 46 and drives the focus lens in the determined direction. If the camera CPU 46 determines that the AF evaluation value signal received continuously from the contrast AF circuit 19 is a turning value under prescribed conditions at a certain point in time, the camera CPU 46 determines focus to be achieved at that point in time. When the focus is determined, a focus lens drive completion signal is transmitted to the lens CPU 7 and the lens CPU 7 stops the driving of the focus lens in response to this signal.

When the focus lens drive is begun at step S102, the camera CPU 46 acquires the AF evaluation value signal in the manner described above at step S103. The camera CPU 46 then evaluates the acquired AF evaluation value signal and works together with the lens CPU 7 to suspend driving of the focus lens.

At step S104, the camera CPU 46 acquires the biometric information of the photographer from at least one of the camera-side biosensor sections 16 and the lens-side biosensor sections 8. At step S105, the camera CPU 46 compares the acquired biometric information to previously acquired biometric information, and determines whether there has been a change. In particular, the camera CPU 46 detects whether the emotional state of the photographer has changed from a normal state to an agitated state, i.e. an emotionally unstable state.

The biometric information for the photographer in the normal state is accumulated in the flash ROM 39. When the photographer uses the camera system 1, the camera CPU 46 periodically and intermittently acquires the biometric information of the photographer and accumulates, as the biometric information of the normal state, biometric information within a prescribed range in which the output of the sensors is stable. Accordingly, the camera CPU 46 can estimate whether the photographer is currently in the normal state by comparing the acquired biometric information to the biometric information of the normal state accumulated in the flash ROM 39. The camera CPU 46 can also determine if the photographer is in an agitated state by comparing the acquired biometric information to the biometric information of the normal state accumulated in the flash ROM 39. For example, if the output indicates that the heart rate is high and the amount of sweat is irregular compared to the normal state, the camera CPU 46 can determine that the photographer is in an agitated state.

If it is determined at step S105 that there is no change, the process moves to step S106 and the camera CPU 46 achieves the focused state and determines that the films lens driving may be ended. If the camera CPU 46 determines that focus has not yet been achieved and that the focus lens driving is to continue, the process moves back to step S102 and the focusing operation continues.

No change in the biometric information at step S105 is estimated to mean that the photographer checking the focus operation while viewing the live view image is satisfied with the focusing operation. When it is affirmatively determined that the focusing operation is satisfactory, a prescribed emotion may be estimated based on the biometric information.

As described above, the lens-side biosensor sections 8 and camera-side biosensor sections 16 are formed as integrated bodies including a variety of sensors, and each sensor outputs a different type of biometric information. By malting a determination based on these individual outputs or a combination of the outputs, certain emotions of the photographer can be estimated. For example, when a high heart rate and emotional swear are detected, it can be estimated that the photographer is feeling “impatient.” The relation between the sensor output and the emotion is obtained verifiably, and a correspondence table can be stored in the flash ROM 39. When estimating the emotion, a determination is made as to which prescribed emotion pattern recorded in the table matches the acquired biometric information.

When the camera CPU 46 reaches the focused State and finishes driving the focus lens at step S106, the process moves to step S107. At step S107, in order to make the photographer aware that the focusing has finished, the camera CPU 46 superimposes a green rectangle, for example, that surrounds the focal point detection region on the live view image.

After the photographer is made aware of the focused state at step S107, the camera CPU 46 performs biometric information detection again at step S108. At step S109, the camera CPU 46 again checks whether there has been a change in the biometric information. In other words, the camera CPU 46 detects whether the photographer feels agitated about the focused state.

When it is determined at step S109 that there is no change in the biometric information, the camera CPU 46 ends the series of autofocus control operations. After the autofocus control ends, the camera system 1 waits until the release SW 24 is fully pressed, and then begins the image capturing operation to generate a subject image.

When there is a change at step S105, the camera CPU 46 moves to step S110. Here, a change in the biometric information is estimated to mean that the photographer checking the focus operation while viewing the live view image is not satisfied with the focusing operation. For example, the photographer might be agitated because, despite wanting to focus on a closer subject, the continuously displayed live view image gradually focuses on subjects that are farther away. The camera CPU 46 obtains this change in the emotional state from at least one of the lens-side biosensor sections 8 and the camera-side biosensor sections 16. The camera CPU 46 may more positively determine that the photographer is in an agitated state in step S105 based on the emotion estimation.

Steps S110 to S115 are performed when it is determined that the photographer feels agitated during the focus lens driving. First, at step S110, the camera CPU 46 changes the focus lens driving operation. The changed focus lens driving operation can have a variety of operational settings. For example, in a case where the photographer wants to focus on a closer subject but the focus lens is driven to focus on subjects that are farther away, the camera CPU 46 preferably reverses the drive direction in order to focus on the closer subject.

A microphone for picking up the voice of the photographer is provided on the rear surface of the camera system 1, e.g. near the rear surface monitor 37, a dictionary (ROM) storing a plurality of key words is provided, and words relating to the operational speed, such as “slow,” “too slow,” “fast,” and “too last” are registered as the key words. When the photographer wants to focus more quickly and says the word “slow,” the camera CPU 46 increases the driving speed of the focus lens. On the other hand, when the driving speed of the focus lens is too fast and the photographer says the word “fast,” the camera CPU 46 decreases the driving speed, or may temporarily stop the focusing depending on circumstances. In the present embodiment, the camera CPU 46 detects when the photographer is in a state other than the normal state, e.g. an agitated state, based on at least one of the lens-side biosensor sections 8 and camera-side biosensor sections 16, and changes the driving speed of the drive apparatus when a key word relating to operating speed is received by the microphone that picks up the voice of the photographer. Therefore, the driving speed of the drive apparatus can be changed according to the state of the photographer.

The camera CPU 46 may also use change in the output of the pressure sensor 23 that detects the pressure with which the camera body 2 is held. For example, the camera CPU 46 may increase the driving speed of the drive apparatus when the output of the pressure sensor 23 indicates that the photographer is holding the camera body 2 less strongly than usual, and may decrease the driving speed of the drive apparatus when the output of the pressure sensor 23 indicates that the photographer is holding the camera body 2 more strongly than usual. In this case, the output of the microphone described above may also be used, but is not necessary.

In a case where the photographer wants to focus on the eyes of a person serving as the subject but the drive of the focus lens results in focusing on the nose, for example, the focal point detection region can be changed from the nose to the eyes. At this time, the camera CPU 46 can judge where to change the focal point detection region to by considering color information in and around the focal point detection region. The camera CPU 46 can change the focal point detection region based on the direction in which voices are input to the microphone 42, according to the condition of the subjects. For example, the camera CPU 46 can reset the focal point detection region in a direction from which the loudest voice is heard.

The camera system 1 includes a plurality of focal point adjustment modes, and the camera CPU 46 can change the drive operation of the focus lens according to the set focal point adjustment mode. For example, when a continuous AF mode is set that continuously matches the focal point to the movement of a subject, if the focus lens is being driven to focus on a subject B who is not moving despite the photographer wanting to match the focal point to subject A who is moving, the focal point detection region is preferably changed to the region of subject A. The camera CPU 46 can also reference the color information of the subject when changing the focal point detection region to a moving subject.

At step S110, the determination about how to change the drive operation of the focus lens is made by sequential testing according to a predetermined priority order. At this time, the priority order may be changed based on the emotion of the photographer estimated from the biometric information acquired at step S104. Furthermore, a configuration may be used in which the photographer can set in advance what changes are made to the focus lens driving.

According to the focus lens driving operation changed at step S110, the camera CPU 46 drives the focus lens via the lens CPU 7 at stop S111. The process then moves to step S112, where the camera CPU 46 determines if the focused state has been achieved and the driving of the focus lens can be stopped. When it is determined that the focused state has been reached, the camera CPU 46 ends the driving of the focus lens, and proceeds to step S107. When it is determined that the focused state has not yet been reached and that driving of the focus lens should continue, the camera CPU 46 moves to step S113.

At step S113, the camera CPU 46 determines whether a prescribed time has passed from when the focus lens driving operation was changed at step S110. The prescribed time is set to be approximately a time in which the emotion of the photographer can change. For example, the time needed to return to a normal state from an agitated state. If the prescribed time has not passed, the camera CPU 46 returns to step S111 and continues to drive the focus lens.

If the prescribed time has passed, at step S114, the camera CPU 46 again acquires the biometric information of the photographer from at least one of the camera-side biosensor sections 16 and lens-side biosensor sections 8. At step S115, the camera CPU 46 determines whether the photographer has left the agitated state. If it is determined that the photographer is no longer in the agitated state, the process moves to step S106. If it is determined at step S106 that the driving of the focus lens is not finished, the camera CPU 46 continues the focusing operation according to the focus lens driving operation changed at step S110. If it is determined that the photographer is still in the agitated state, the camera CPU 46 returns to step S110. At step S110, a change is made to another focus lens driving operation.

When it is determined at step S109 that there is a change in the biometric information, the camera CPU 46 proceeds to step S116. A change in the biometric information at step S109 can be estimated as being caused by the photographer being agitated about the focus state as a result of viewing the superimposed display. Therefore, at step S116, the camera CPU 46 changes the focal point detection region. At this time, the camera CPU 46 determines, as a new focal point detection region, a face region that differs from the fact region as the focal point detection region previously at step S101. When the new focal point detection region is set, the camera CPU 46 returns to step S102 and continues the focusing operation.

The flow described above is an example in which the focal point detection region is determined based on recognition of a face of a subject by the face recognition section 45, but the determination of the focal point detection region is not limited to this. The photographer may select any region, or the focal point detection region may be set as a region corresponding to a subject positioned near the center of the angle of field or the subject closest to the camera.

In the above flow, the focusing operation was performed by contrast AF, but a phase difference AF using the focal point detection sensor 29 can be applied instead. In a case where the photography lens 3 is a telescopic lens and the a repeating scan operation is performed for the focus lens in one prescribed direction to find the focal position when the focal point has a large skew, the photographer could experience a disorienting feeling when the prescribed direction is opposite the focusing direction. In such a case, the camera CPU 46 detects the change in the biometric information of the photographer, e.g. agitation or a feeling of disorientation, and may change the direction of the focus lens scanning operation to be the opposite direction. In this case, the camera CPU 46 suitably controls the phase difference AF by selecting the focal point detection region, using the focus lens driving operation, or the like. For example, when color information of the subject is used in the focus lens driving operation, the output of the photometric sensor 40 can be used.

Japanese Patent Application Publication No. 2007-233032 (US Patent Application Publication No. 20070206937) proposes, as a phase difference AF, an image capturing element AF in which pixels for AF detection are provided in the image capturing element to perform a phase difference AF. This type of image capturing element AF may be applied to the first embodiment example described above.

### Second Embodiment Example

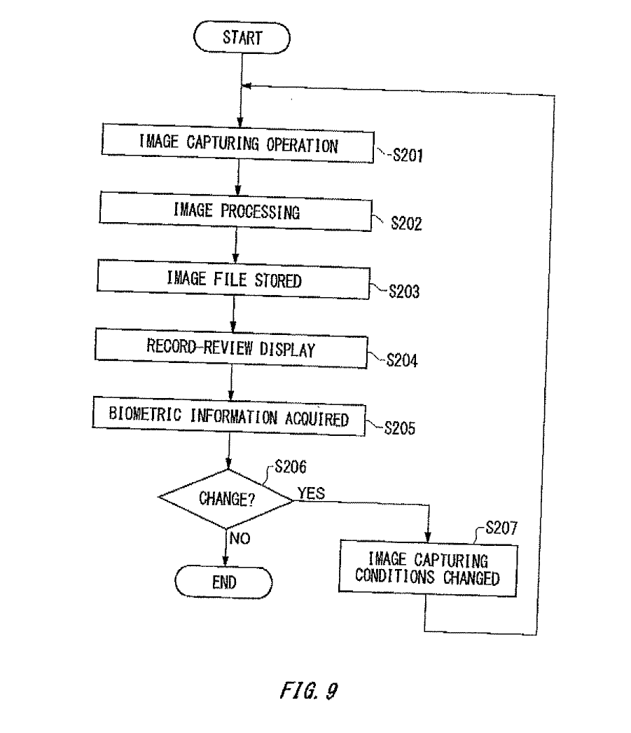

FIG. 9 shows a flow for controlling image capturing as a second embodiment example of the present embodiment. In the present embodiment example, a still image is captured. The camera CPU 46 performs an image capturing preparation operation such as light measurement or autofocus when the photographer presses the release SW 24 half way to turn SW1 ON. The release SW 24 is then fully pressed to turn SW2 ON, thereby initiating the image capturing operation of step S201. In the image capturing operation, according to the determined exposure value, the lens CPU 7 operates the diaphragm 5 and the camera CPU 46 moves the focal plane shutter to guide subject light to the image capturing element 27. Furthermore, according to the determined image capturing sensitivity, the camera CPU 46 applies a prescribed gain to the output of the image capturing clement 27 and reads the charge. At step S202, the image processing control circuit 18 generates an image file by applying image processing and a compression process to the image signal generated as described above.

The image file generated at step S202 is stored in the image recording medium 35 at step S203. At step S204, the image-processed image data is displayed in the rear surface monitor 37 by the rear surface monitor control section 36 for a set prescribed time, e.g. approximately three seconds. The photographer can recognize the image immediately after capture using record-review.

The record-review recognition of the photographer is obtained and, at step S205, the camera CPU 46 obtains the biometric information of the photographer from at least one of the camera-side biosensor sections 16 and the lens-side biosensor sections 8. At step S206, the camera CPU 46 compares the acquired biometric information to previously acquired biometric information, and determines whether there has been a change. In particular, the camera CPU 46 detects whether the emotional state of the photographer has changed from a normal state to an agitated state or a dismayed state.

When it is determined at step S206 that there is no change, the camera CPU 46 determines that the photographer is satisfied with the image capturing results and ends the series of image capturing operations. On the other hand, if it is determined that the biometric information of the photographer has changed, the camera CPU 46 determines that the photographer is not satisfied with the image capturing results, and moves to step S207.

The camera CPU 46 changes the image capturing conditions to again perform image capturing at step S207. In the present embodiment, a change in the exposure value is used as the change in the image capturing conditions. The exposure value is defined by three values corresponding to the exposure time during which the image capturing element 27 is exposed to subject light, and the diaphragm value of the diaphragm 5 limiting the subject light, the output gain of the image capturing element 27, but these values are changed from the exposure value applied during the previous image capturing. Furthermore, the exposure value is changed to be a prescribed number of levels above or below the exposure value calculated from the output of the photometric sensor 40, and a gray scale correction process is applied to the acquired image. In this way, the setting may be changed to apply active D lighting that maintains the tone and realizes overall suitable exposure. Furthermore, the camera-shake correction described above may be set automatically. The image capturing mode can also be changed. The camera CPU 46 can switch from a diaphragm priority mode to a shutter speed priority mode, for example, or can switch from a close-up mode to a scenery mode. When the image capturing conditions are changed at step S207, the camera CPU 46 returns to step S201 and begins the image capturing operation again.

In the flow described above, the process proceeds to step S205 and the biometric information is acquired automatically after the record-review display is performed at step S204, but the acquisition of the biometric information may be performed on a condition that the captured image is deleted by instructions from the photographer within a prescribed time after the record-review display. As a result of the photographer operating the operation SW, the camera CPU 46 can delete an image file stored in the image recording medium 35, and if the captured image file is deleted within a prescribed time after the image capturing, there is a high probability that the photographer felt agitated by the image capturing, particularly when a plurality of image files are deleted within a prescribed time after image capturing. Therefore, if a captured image file is deleted within a prescribed time after image capturing, if the camera CPU 46 acquires the biometric information of the photographer and estimates that the photographer is agitated based on a change in the biometric information, the camera CPU 46 automatically restarts the image capturing operation.

There are cases in which the operation SW is unintentionally operated to mistakenly delete an image file. In such a case, the camera CPU 46 can cancel the deletion of the image file if the biometric information of the photographer or operator has changed greatly. In this case, a temporary deletion holder is provided and the camera CPU 46 preserves the image file that was designated for deletion temporarily in the temporary deletion holder, and may then delete the image after confirming that the biometric information of the photographer or the operator has not changed greatly after a prescribed time, e.g. two or three seconds, has passed. In addition, a microphone that picks up the voice of the photographer, such as in the first embodiment example, may be provided and words such as “darn” or “oops” that are commonly said when a mistake is made may be registered in the dictionary (ROM) as key words. When the microphone receives a voice indicating a mistaken operation, the camera CPU 46 may determine that the operation performed immediately therebefore by the photographer was a mistake, and cancel or suspend that operation. In this case, the camera CPU 46 may detect a change in the biometric information of the photographer or operator and determine that there was a mistaken operation. Similarly, when the power supply of the camera system 1 is mistakenly turned OFF, if there is a change in the biometric information or the voice picked up by the microphone indicates a mistaken operation, the camera CPU 46 may ignore the operation of turning the power supply OFF.

An image capturing section for capturing an image of the photographer may be provided near the rear surface monitor 37, e.g. above the rear surface monitor 37, to detect the facial expression of the photographer. For example, an image of the eyebrows of the user can be captured by capturing images of the left eye and right eye of the photographer, and the photographer may be determined to be agitated when a furrow is detected in the brow. The detection of a furrow in the brow may be achieved by pattern matching with an image of a furrowed brow stored in the flash ROM 39 as a reference image, or by detecting a shadow distribution between the left and right eyes. US Patent Application Publication No. 2008-292148 describes detection of a furrowed brow. The state of the photographer can be more accurately determined if the expression detection results described above are used in addition to the output from the lens-side biosensor sections 8 and the camera-side biosensor sections 16.

The first and second embodiment examples described above are examples of an image capturing operation for a still image. However, the image capturing can be controlled based on the biometric information detection results for a moving image as well. For example, since contrast AF is also performed when capturing a moving image, the focus lens driving operation can be changed based on change in the biometric information in the same manner as in the first embodiment example.

The first and second embodiment examples described above are examples in which the camera CPU 46 compares acquired biometric information to previously acquired biometric information to determine if there is a change. Instead, the camera CPU 46 may determine a change in the photographer when the biometric information indicates a large change, such as a change of 10% or more.

In the above embodiments, the camera body 2 and the photography lens 3 are respectively provided with the lens-side biosensor sections 8 and the camera-side biosensor sections 16. However, the biosensors may be formed independently and attached more directly to the body of the photographer. For example, a biosensor formed as a wrist watch, such as described in Japanese Patent Application Publication No. 2005-270543 (U.S. Pat. No. 7,538,890), may be used. In this case, the camera system 1 may include a biometric information acquiring section that uses a wire or that is wireless.

In the above embodiments, there is no specific description about the power supply, but it is obvious that the camera system 1 operates by receiving power. The camera system 1 can continuously receive power from a domestic AC power supply, and can also receive power from a detachable battery. The battery may be one-dimensional or two-dimensional.

A plurality of batteries can be attached and detached according to the properties of the device that supplies power. For example, when the camera body 2 and the photography lens 3 are formed as separate units, as in the above embodiment, a battery may be equipped in each unit. In this case, the battery equipped in the camera body 2 may provides power primarily to the camera body 2, and the battery equipped in the photography lens 3 provides power primarily to the photography lens 3. Accordingly, the drive power for driving the focus lens is supplied by the battery equipped in the photography lens 3. Of course, when a battery is empty, for example, one of the batteries can supply power to compensate for the empty battery.

In the above embodiments, the camera system 1, which is a single lens reflex camera with an exchangeable lens, is provided as an example of the electronic device, but the present invention is not limited to the camera system 1. The present invention can also be applied to a compact digital camera, a mirrorless single lens camera, a mobile phone, a video camera, or any electronic device in which the processing can be changed according to change in biometric information of the user.

While the embodiments of the present invention have been described, the technical scope of the invention is not limited to the above described embodiments. Furthermore, the first and second embodiment examples can be combined. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### DESCRIPTION OF THE REFERENCE NUMERALS

1: camera system, 2: camera body, 3: photography lens, 4: lens group, 5: diaphragm, 6: angular velocity sensor, 7: lens CPU, 8: lens-side biosensor section, 9: heart rate detection apparatus, 10: drive circuit, 11: A/D conversion circuit, 12: pulse detection apparatus, 13: sweat sensor, 14: temperature sensor, 15: pressure sensor, 16: camera-side biosensor section, 17: heart rate detection apparatus, 18: image processing control circuit, 19: contrast AF circuit, 20: pulse detection apparatus, 21: sweat sensor, 22: temperature sensor, 23: pressure sensor, 24: release SW, 25: image capturing mode SW, 26: finder optical system, 27: image capturing element, 28: main mirror, 29: focal point detection sensor, 30: sub-mirror, 31: focusing screen, 32: pentaprism, 33: low-pass filler, 34: image capturing substrate, 35: image recording medium, 36: rear surface monitor control section, 37: rear surface monitor, 38: calendar section, 39: flash ROM, 40: photometric sensor, 41: GPS module, 42: microphone, 43: speaker, 44: RAM, 45: face recognition section, 46: camera CPU

1. An electronic device comprising:
a processing section that performs processing; and
a control section that changes the processing by the processing section upon receiving a change in biometric information of a user caused by the processing of the processing section.

2. The electronic device according to claim 1, comprising a sound input section that inputs sound, wherein
the control section changes the processing of the processing section when the sound input section inputs a sound caused by the processing of the processing section.

3. The electronic device according to claim 1, comprising an expression detecting section that detects an expression of the user, wherein
the control section changes the processing of the processing section according to a detection result of the expression detecting section.

4. The electronic device according to claim 1, comprising an input section that inputs a change in the biometric information of the user.

5. The electronic device according to claim 1, wherein
the processing section includes an image capturing section that captures an image via an optical system including a focus lens, and focal point detecting section that detects a focal point of the optical system, and
the control section changes drive control of the focus lens according to change in the biometric information of the user when driving the focus lens based on a detection result of the focal point detecting section.

6. The electronic device according to claim 5, wherein
the control section stops the driving of the focus lens or reverses a direction of the driving.

7. The electronic device according to claim 5, wherein
the control section changes driving speed of the focus lens.

8. The electronic device according to claim 5, wherein
the control section changes the drive control of the focus lens by considering color information of an adjustment region in which the focal point is adjusted.

9. The electronic device according to claim 5, wherein
the control section changes the drive control of the focus lens based on a direction of sound input by the sound input section.

10. The electronic device according to claim 5, wherein
the control section changes the drive control of the focus lens according to a set focal point adjustment mode.

11. The electronic device according to claim 5, wherein
the processing section includes a face recognition section that recognizes faces of a plurality of subject people, and
the control section changes a face for which focal point adjustment is performed from among the plurality of faces detected by the face recognition section.

12. The electronic device according to claim 1, wherein
the processing section includes an image capturing section that captures an image via an optical system having a focus lens, a setting section that sets image capturing conditions for the image capturing section, and a display section that displays the captured image, and
the control section changes the image capturing conditions set by the setting section, according to a change in the biometric information of the user when the image is displayed in the display section.

13. The electronic device according to claim 12, wherein
the processing section includes a deleting section that deletes the captured image, and
the control section changes the image capturing conditions set by the setting section, according to a change in the biometric information of the user when the image is deleted by the deleting section.

14. The electronic device according to claim 12, wherein
the control section changes at least one of exposure time, diaphragm value, image capturing sensitivity, and image capturing mode.

15. The electronic device according to claim 5, comprising a power supply section that is provided in an optical unit of the optical system and supplies power at least to the optical unit.

16. The electronic device according to claim 1, comprising a process setting section that sets the process to be performed by the processing section, wherein
the control section stops the processing by the processing section, at least temporarily, when a change in the biometric information of the user is received after the setting by the process setting section.
